(19) United States
(12) Patent Application Publication
(10) Pub. No.: US 2006/0193894 A1
(43) Pub. Date: Aug. 31, 2006

(54) Methods for providing biomedical devices with hydrophilic antimicrobial coatings

(76) Inventors: James S. Jen, Jacksonville, FL (US); Robert E. Jones, Jacksonville, FL (US); Pastora M. Homesley, Fernandina Beach, FL (US); James Petisce, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/068,008

(22) Filed: Feb. 28, 2005

(51) Int. Cl. A61L 33/00; B05D 3/02

(52) U.S. Cl. 424/427; 427/2.24

(57) ABSTRACT

The invention provides a process for producing devices with stable surface coatings, which coatings are one or both of hydrophilic and antimicrobial.

### FIELD OF THE INVENTION

This invention relates to coated devices. In particular, the invention provides methods for coating biomedical devices with stable, hydrophilic, antimicrobial coatings.

### BACKGROUND OF THE INVENTION

Devices for use in and on the human body are well known. The chemical composition of the surfaces of such devices plays a pivotal role in dictating the overall efficacy of the devices. For example, many devices, including catheters, stents, contact and intraocular lenses, and implants require biologically non-fouling surfaces, meaning that proteins, lipids, and cells will not adhere to the surface. Contact lenses also must be wettable by tear fluid in order to ensure wearer comfort. Additionally, providing such devices with an antimicrobial surface is advantageous, especially in extended wear contact lenses.

A wide variety of methods have been developed to coat device surfaces to provide them with desired characteristics. For example, it is known to coat contact lenses with hydrophilic and anti-microbial coatings by soaking the lenses in the coating materials or incorporating the materials into the lens material. However, these methods are disadvantageous in that the coatings tend to leach from the lens over time.

### DETAILED DESCRIPTION OF THE INVENTION AND PREFERRED EMBODIMENTS

The present invention provides a simple, economical process for producing devices with stable surface coatings, which coatings are one or both of hydrophilic and antimicrobial. By “antimicrobial” is meant that bacterial adherence to the device surface is reduced in comparison to the uncoated surface, by about 97 percent or more.

In one embodiment, the invention provides a method for manufacturing biomedical devices comprising, consisting essentially of, and consisting of (a.) contacting at least one surface of a biomedical device with a coating effective amount of a humectant and (b.) irradiating the device and humectant under conditions suitable to produce a stable coating on the surface wherein the coating is hydrophilic, antimicrobial, or both. In another embodiment, the invention provides biomedical devices manufactured according to the method of the invention.

By “biomedical device” is meant any device designed to be used while in or on either or both human tissue or fluid. Examples of such devices include, without limitation, stents, implants, catheters, and ophthalmic lenses. In a preferred embodiment, the biomedical device is an ophthalmic lens including, without limitation, contact, intraocular lenses onlay lenses and the like. More preferably, the device is a contact lens.

By “stable coating” is meant that subjecting the coating to one or more of autoclaving, washing with a cleaning agent, or rinsing with a saline solution does not substantially alter the chemical properties of the coating. By “humectant” is meant an agent that lowers the total free energy of water and is capable of binding water.

It is an unexpected discovery of the invention that stable coatings may be formed that are either or both hydrophilic and antimicrobial by use of a humectant and irradiation. Thus, in the first step of the invention, the device is contacted with a humectant. Contacting may be carried out by any convenient method including, without limitation, soaking, spraying, coating or a combination thereof Preferably, contacting is carried out by soaking or spraying, more preferably by soak coating.

The specific humectant selected, the amount used, and the time for contacting will depend upon the material from which the device is formed. Suitable humectants include, without, limitation, polymeric humectants and non-polymeric humectants. The polymeric humectants include, without limitation, hydroxyethyl acrylate (“HEA”), 2-hydoxyethyl methacrylate (“HEMA”), dimethacrylamide (“DMA”), polyvinyl alcohol, polyvinyl pyrrolidone, polyethylene glycol (“PEG”), di(ethylene glycol)vinyl ether (“EO₂V”), cellulose derivatives, and the like and combinations thereof. Non-polymeric humectants include, without limitation, glycerin, urea, propylene glycol, non-polymeric diols, glycerols, and the like and combinations therefor. In embodiments in which the device is a contact lens, preferably the humectant is HEA or a polyethylene glycol. For lenses that are high Dk, for the purposes of this invention meaning a Dk of 60 or greater, silicone hydrogel lenses, the humectant preferably is HEA.

The amount of humectant used will be a coating effective amount. By a coating effective amount is meant an amount sufficient to coat the surface to the desired degree. Conveniently, an aqueous solution of the humectant is used in which the amount of humectant used is about 0.05% to about 10%, preferably about 0.1% to about 5%, more preferably, about 0.2% to about 1% weight percent of the solution. One ordinarily skilled in the art will recognize that the humectant solution may contain additives including, without limitation, initiators, processing aids and the like.

One or more surfaces of the device may be coated using the process of the invention. Preferably, the surface is made of a silicone elastomer, hydrogel, or silicone-containing hydrogel. More preferably, the surface is a siloxane including, without limitation, polydimethyl siloxane macromers, methacryloxypropyl polyalkyl siloxanes, and mixtures thereof, silicone hydrogel or a hydrogel. More preferably, the surface is made of etafilcon, galyfilcon, lenefilcon, or senefilcon.

In contacting of the device with the humectant, temperature and pressure are not critical and the process may be conveniently carried out at room temperature and pressure. The contact time used will be a length of time sufficient to coat the surface to the extent desired. Generally, contact times will be from about 10 seconds to about 2 hours, preferably from about 5 seconds to about 1 hour.

Following the contacting step, the humectant coated device is irradiated. Any suitable irradiation source may be used, but preferably an ultraviolet light source is used. Irradiation times will vary depending upon the device being coated and the humectant selected. Preferably, irradiation is carried out for a total of about 1 second to about 15 minutes, more preferably 3 seconds to about 10 minutes, most preferably 15 seconds to about 5 minutes. Following irradiation, the surface may be washed with water or buffered saline solution to remove unreacted humectant and additives.

Preferably, an initiator is used in the humectant solution. The initiator selected will depend upon the type of irradiation selected. For example, when UV irradiation is used, UV suitable initiators are DAROCUR™ 1173, IRGACURE™ 819, IRGACURE™ 1850, and the like and combinations thereof. Typically, the UV initiator will be used in an amount of about 0.2 to about 1 weight percent.

The invention will be further clarified by a consideration of the following, non-limiting examples.

### EXAMPLES

### Example 1

Silicone lenses were made according to formulation 8 of Table 1 of U.S. Pat. No. 6,367,929 B1, incorporated herein in its entirety by reference. The lenses were then immersed in approximately 3 ml of HEA, which amount was sufficient solution to allow the lenses be totally immersed, for approximately 15 minutes followed by immersion for 3 seconds after 0.2% wt. of an initiator, DAROCUR™ 1173, was added. Immersion was carried out at the room temperature (approximately 22° C.) and ambient conditions. The lenses were then removed from the solution and irradiated using a Dymax 2000EC ultraviolet light with a 400-watt metal halide lamp that produces 100 mW/cm². The distance between the lamp and the sample was approximately 18 cm and illumination was carried out for approximately 3 seconds.

The lenses were washed 2 times with deionized (“DI”) water and then soaked in 10 ml of DI water for approximately 2 hours. The lenses were stored in a packing solution for testing, which solution was a 0.85% NaCl saline solution buffered with sodium borate and boric acid. The lenses were stored at the room temperature and the storage time varied.

Five of the lenses were soaked overnight at room temperature and pressure in 10 ml of a protein solution containing 1.95 g albumin, 0.60 g lysozyme and 0.80 g immunoglobin in 500 ml. saline solution. The lenses were removed from the protein solution and studied using the Attenuated Total Reflection (“ATR”) technique. One lens removed from the protein solution was initially studied using Fourier Transform Infrared-Attenuated Total Reflection (“FTIR-ATR”) technique. The same lens was washed with DI water for 10 seconds and then again studied using the FTIR-ATR technique. The trace data for all of the lenses showed that, after washing with DI water, the lenses' surfaces were substantially identical to that of the lenses that were not soaked in the protein solution demonstrating that the proteins did not become tightly absorbed to the treated lenses.

The protein solution soaked and then washed lenses, along with lenses that were not soaked in the protein solution, were further tested for contact angles using the Wilhelmy plate method whereby the lenses were suspended on a micro-balance and was immersed in and then pulled out of the packing solution set forth above. Wetting force measured by a micro-balance was used to calculate the contact angle according to the formulation F=γpCOSθ, wherein F was the wetting force, γ was the surface tension of the packing solution, p was the perimeter of the lens and θ was the contact angle. The results are shown on Table 1 below and show that there was no significant difference between the contact angles of the two samples indicating that the proteins were not absorbed onto the soaked lens.

TABLE 1

| | Contact Angle (degrees) | Std. Dev. |
|---|---|---|
| Un-soaked Lenses | 59 | 7 |
| Soaked and Washed Lenses | 51 | 2 |

### Examples 2-5

Example 1 was repeated except that, in place of HEA, some of the lenses were soaked in 1.5 ml EO₂V for 60 minutes, 3 ml of MC PEG 350 for 60 minutes, 3 ml of HEMA for 15 minutes, or 3 ml of DMA for 15 minutes and no DAROCUR was used. The lenses were then irradiated as follows: the EO₂V soaked lenses were UV irradiated for 5 minutes, the MC PEG 350 soaked lenses for 5 minutes, the HEMA soaked lenses for 3 seconds and the DMA soaked lens for 10 minutes.

Subsequently, all lenses were treated and tested the same way as in Example 1. The results are shown in Table 2 below.

TABLE 2

| | Contact Angle (degrees) | Std. Dev. |
|---|---|---|
| Un-soaked Lenses | 91 | 6 |
| DMA Soaked and | 55 | 7 |
| Washed Lenses | | |
| HEMA Soaked and | 73 | 5 |
| Washed Lenses | | |
| EO₂V Soaked and | 61 | 3 |
| Washed Lenses | | |

### Example 6

Silicone hydrogel lenses of the formulation of Example 1 were soaked in HEA solutions of varying concentrations. Lenses were soaked in 20%, 80% or 100% weight percent HEA solutions with 0.2% wt. Darocur added. The lenses were totally immersed in approximately 3 ml of their respective solutions for 15 minutes. UV irradiation was carried out as in Example 1 for 3 seconds, the lenses were washed 2 times with DI water, soaked in 10 ml DI water for 2 hours and then stored in a packing solution glass vial for future testing.

The lenses were then removed from the packing solution and then the contact angles were tested as set forth in Example 1. The results are shown in Table 3 below.

TABLE 3

| | Contact Angle (degrees) | Efficacy (log reduction) |
|---|---|---|
| Uncoated Lens | 71 (std. dev. 3) | |
| 20% HEA | 74 (std. dev. 2) | −0.13 |
| 80% HEA | 64 (std. dev. 2) | 0.46 |
| 100% HEA | 55 (std. dev. 7) | 1.59 |

The results demonstrate that the effect of HEA is concentration dependent; the higher the HEA concentration, the lower the contact angle.

### Example 7

Silicone hydrogel lenses of the formulation of Example 1 were irradiated as in Example 1 except that irradiation was carried out while the lens was being coated using a spray nozzle filled with 100% EO₂V. The UV light and the spray of 3 μl/min were turned on for 15 seconds and then turned off. The lens was then turned over to expose the opposite side of the lens and the procedure was repeated. The lens was then washed with DI water and then stored in packing solution.

### Example 8

Silicone hydrogel lenses of the formulation of Example 1 were individually treated using a 12-well cell culture cluster tray. Approximately 1.5 to 3 ml solution was placed in each well and then one lens was added into each well. Each lens was placed in the EO₂V for 15 minutes and then placed under a Dymax 2000EC UV light with a 400-watt metal halide lamp producing 100 mW/cm² at a distance of 18 cm between the lamp and the lens. The lens was then washed ×2 with DI water and stored in packing solution.

The lenses of Example 7 and 8 were evaluated using contact angle testing as in Example 1. The results of the contact angle testing are shown in Table 4. Contact angles were significantly decreased as compared to that of same lens that was uncoated.

TABLE 4

| | Spray Coated | Soak Coated | Uncoated |
|---|---|---|---|
| Average | 57 | 50 | 91 |
| Std Dev. | 5 | 7 | 6 |

Some of the lenses were then digitally rubbed for 10 seconds Using RENU™ Multiplus cleaning solution. The contact angles were again measured and the results are shown in Table 5.

TABLE 5

| | Spray Coated | Soak Coated | Uncoated |
|---|---|---|---|
| Average | 59 | 75 | 87 |
| Std Dev. | 6 | 4 | 7 |

Other of the lenses were autoclaved at 131° C. for 30 minutes and the contact angles were tested. The results, shown in Table 6, demonstrate that the EO₂V coating remained intact following autoclaving.

TABLE 6

| | Spray Coated | Soak Coated | Uncoated |
|---|---|---|---|
| Average | 64 | 52 | 91 |
| Std Dev. | 12 | 10 | 4 |

### Example 9

Lenses were prepared and tested as in Examples 7 and 8 except that, 3 ml PEG 350 was used in place of the EO₂V. The data on Tables 7, 8, and 9 below show the contact angle data.

TABLE 7

| | | Soak PEG350 | Soak PEG350 |
|---|---|---|---|
| | Soak PEG350 | Coated; Post | Coated; |
| | Coated | digital rub | Post autoclave |
| Average | 60 | 59 | 57 |
| Std Dev. | 5 | 5 | 8 |

TABLE 8

| | | Uncoated; Post | Uncoated; |
|---|---|---|---|
| | Uncoated | digital rub | Post autoclave |
| Average | 91 | 87 | 91 |
| Std Dev. | 6 | 7 | 4 |

TABLE 9

| | PEG350 Soak | | PEG350 Soak | Uncoated |
|---|---|---|---|---|
| | Coated | Uncoated | Coated FOCUS | FOCUS |
| | ACUVUE | ACUVUE | Night & Day | Night & Day |
| Average | 75 | 82 | 55 | 62 |
| Std. Dev. | 3 | 7 | 11 | 7 |

### Example 10

A culture of pseudomonas aeruginosa, ATCC # 15442 (from ATCC, Rockville, Md.) was grown overnight in 150 ml tryptic soy broth. A standardized phosphate buffered saline (“PBS”) washed bacterial inoculum was prepared containing 1×10⁸ cfu/ml. The bacteria were applied to the silicone lenses of the formulation of Example 1, some of which lenses were uncoated and some of which were coated with HEA. The contact lenses were washed with PBS. Each washed lens was combined with 1 ml of the standardized bacterial inoculum in a glass vial, which vial was shaken at 100 rpm in a rotary shaker-incubator for 24 hrs at 35° C. Following the incubation period the lenses were washed 3 times in sterile PBS. Each washed lens was placed into a macerate tube containing one ml of PBS containing 0.05 percent TWEEN™-80 and macerated at a power setting of 3-4 for approximately 10-15 seconds. The resulting macerate as well as the bacterial suspension were enumerated for viable bacteria. The results show that the HEA coating greatly reduced adhesion of bacteria to the lenses. The results are shown in Table 10 below.

TABLE 10

| | | | Log |
|---|---|---|---|
| | Lens | Solution | Reduction |
| HEA Soak + UV Lens | 4.0 × 10⁴ | 4.7 × 10⁶ CFU/ml | None |
| Uncoated lens | 5.1 × 10⁴ | 3.5 × 10⁶ CFU/ml | None |
| HEA Soak Lens | 1.0 × 10⁴ | 3.2 × 10⁴ CFU/ml | 1.07 |
| Uncoated lens | 5.5 × 10⁴ | 3.8 × 10⁵ CFU/ml | None |

What is claimed is:

1. A method for manufacturing biomedical devices, comprising the steps of:
(a.) contacting at least one surface of a biomedical device with a coating effective amount of a humectant; and
(b.) irradiating with ultraviolet radiation the device and humectant under conditions suitable to produce a stable coating on the surface wherein the coating is hydrophilic, antimicrobial, or both.

2. The method of claim 1, wherein the device is a contact lens.

3. The method of claim 1, wherein the humectant is a polymeric humectant, a non-polymeric humectant, or a combination thereof.

4. The method of claim 2, wherein the humectant is a polymeric humectant, a non-polymeric humectant, or a combination thereof.

5. The method of claim 1, wherein the humectant is a polymeric humectant selected from the group consisting of hydroxyethyl acrylate, 2-hydoxyethyl methacrylate, dimethacrylamide, polyvinyl alcohol, polyvinyl pyrrolidone, polyethylene glycol, di(ethylene glycol)vinyl ether, cellulose derivatives, and the like and combinations thereof.

6. The method of claim 2, wherein the humectant is a polymeric humectant selected from the group consisting of hydroxyethyl acrylate, 2-hydoxyethyl methacrylate, dimethacrylamide, polyvinyl alcohol, polyvinyl pyrrolidone, polyethylene glycol, di(ethylene glycol)vinyl ether, cellulose derivatives, and the like and combinations thereof.

7. The method of claim 1, wherein the humectant is a non-polymeric humectant selected from the group consisting of glycerin, urea, propylene glycol, non-polymeric diols, glycerols, and the like and combinations therefor.

8. The method of claim 2, wherein the humectant is a non-polymeric humectant selected from the group consisting of glycerin, urea, propylene glycol, non-polymeric diols; glycerols, and the like and combinations therefor.

9. The method of claim 2, wherein the humectant is hydroxyethyl acrylate or a polyethylene glycol.

10. The method of claim 1, wherein the irradiation is carried out for a total of about 1 second to about 15 minutes.

11. The method of claim 2, wherein the irradiation is carried out for a total of about 1 second to about 15 minutes.

12. A contact lens produced by the method of claim 2.

13. A contact lens produced by the method of claim 4.

14. A contact lens produced by the method of claim 6.

15. A contact lens produced by the method of claim 8.

16. A contact lens produced by the method of claim 9.
